(19) United States
(12) Patent Application Publication
(10) Pub. No.: US 2004/0029141 A1
(43) Pub. Date: Feb. 12, 2004

(54) Human and mouse e2-protein nucleic acids coding therefor and uses thereof

(76) Inventors: Peter Brodin, Molndal (SE); Anders Thelin, Molndal (SE)

Correspondence Address: FISH & RICHARDSON PC, 225 FRANKLIN ST, BOSTON, MA, 02110, US

(21) Appl. No.: 10/362,711

(22) PCT Filed: Aug. 23, 2001
(86) PCT No.: PCT/GB01/03807

(30) Foreign Application Priority Data
Aug. 28, 2000 (US) 60228118

(51) Int. Cl. C12Q 1/68; A01K 67/00; C07H 21/04; C07K 14/72; C12P 21/02; C12N 5/06

(52) U.S. Cl. 435/6; 435/69.1; 435/320.1; 435/325; 530/350; 536/23.5; 800/8

(57) ABSTRACT

This invention relates to the regulation of metabolism and in particular to a gene named E2 involved in insulin resistance syndrome. The invention further relates to protein encoded by the gene and to means of regulating their biological activity. In addition the invention relates to the use of the gene and protein to identify therapeutic agents for controlling insulin resistance syndrome and other related disorders such as non-insulin dependent diabetes mellitus (NIDDM), dyslipidemia, obesity and atherosclerosis.

This invention relates to the regulation of metabolism and in particular to genes involved in insulin resistance syndrome. The invention further relates to proteins encoded by the genes and to means of regulating their biological activity. In addition the invention relates to the use of the genes and proteins to identify therapeutic agents for controlling insulin resistance syndrome and other related disorders such as non-insulin dependent diabetes mellitus (NDDM), dyslipidemia, obesity and atherosclerosis.

Insulin resistance syndrome (IRS) is a complex metabolic disorder which initially is associated with elevated levels of insulin. Affected individuals become resistant to the biological action of insulin. In later stages of the syndrome the insulin levels drop and glucose levels rise and the individual will enter a diabetic state (O'Rahilly S., BMJ 1997, 314(7085), 955-959). The drop in insulin levels is probably caused by a collapse of insulin production in the pancreas. The mechanisms behind this disorder are unknown but studies have shown that the development of IRS is multifactorial involving both environmental and genetic components. Obesity is believed to be a major component in the development of IRS and since obesity is increasing in the western world, IRS is also an increasing problem. If untreated IRS will lead to the development of atherosclerosis and premature death.

Current treatment is unsatisfactory and new drugs need to be developed. A major problem is that the mechanisms behind the syndrome are unknown and that no reliable or relevant experimental models for human drug development are available. At present drug research has to rely on a few rodent models with single gene defects in appetite regulation, or high calorie diet treated rodents.

The thiazolidinedione (TZD) class of compounds, used as insulin-sensitising drugs for treatment of NIDDM, has been shown to have desirable effects on levels of plasma glucose, triglycerides and insulin in mice, rats and humans (Young P. W. et al., Diabetes 1995, 44(9), 1087-1092; Shimabukuro M., et al., J. Biol. Chem. 1998, 273(6), 3547-3550; Antonucci T., et al., Diabetes Care 1997, 20(2), 188-193 [published erratum appears in Diabetes Care 1998, 21(4), 678]). Thiazolidinediones bind to the nuclear receptor peroxisome proliferator activated receptor γ (PPAR-γ) (Wiesenberg I., et al., Molecular Pharmacol. 1998, 53(6), 1131-1138). The binding of TZD to PPAR-γ is thought to mediate the effects of these compounds and is likely to affect the transcription of specific genes. However, which these genes are and how they can give beneficial effects on IRS is still unknown.

The present invention relates to our discovery of a previously unknown gene, E2, which is differentially expressed in untreated and rosiglitazone-(TZD X103, BRL49653) treated ob/ob mice. These mice are leptin deficient, obese, and develop a condition resembling NIDDM with age. The identified sequences have been confirmed using real-time quantitative PCR in order to authenticate the differential expression pattern. Confirmed sequences have been further validated in time-course and tissue distribution experiments.

E2 message is up-regulated after rosiglitazone treatment in epididymal fat tissue and also in mesenterial fat tissue. In addition, E2 message levels increase substantially after the first rosiglitazone administration to obese mice and are further increased over a 7-day period. The up-regulation of E2 precedes the effects on plasma glucose and triglycerides which are not lowered by the first administration of rosiglitazone. These results indicate that E2 has a direct role in the regulation of metabolism; and that compounds which modulate the activity of E2 will have utility as therapeutic agents for controlling metabolic disorders, for example insulin resistance syndrome, non-insulin dependent diabetes mellitus, dyslipidemia, obesity and atherosclerosis.

The present invention discloses full length cDNA and protein sequences for both human and mouse E2. E2 cDNA shares sequence homology with a known human sequence, EMBL AF 092133. Human E2 differs from EMBL AF 092133 by a 13 base-pair insertion which is found in EMBL AF 092133 at position 280, so causing a shift in the reading frame. Human E2 codes for a 315 amino acid protein whereas EMBL AF 092133 comprises 204 amino acids. EMBL AF 092133 lacks 111 amino acids in the N-terminal compared to human E2.

The invention further discloses that E2 has utility in the development of new therapeutic agents for use in the treatment of insulin resistance syndrome and other related disorders such as non-insulin dependent diabetes mellitus, dyslipidemia, obesity and atherosclerosis. The invention further provides methods for the identification of such therapeutic agents.

Unless defined otherwise, all technical and scientific terms used herein have the same meaning as is commonly understood by one of skill in the art to which this invention belongs. All publications and patents referred to herein are incorporated by reference.

In a first aspect of the present invention we provide an isolated or purified polynucleotide molecule comprising a nucleic acid sequence which encodes an E2 polypeptide or a polypeptide fragment thereof of greater than 204 amino acids. By the term “isolated”, we mean that the polynucleotide molecule has been separated from those constituents that are normally present with it in nature. Preferably the E2 polypeptide or fragment thereof is selected from:

i) SEQ ID NO: 2 or a fragment thereof selected from SEQ ID NO:2 positions 1-265, 1-270, 1-280, 1-290, 10-315, 15-315, 20-315, 25-315, 35-315, 10-290, 15-285, 20-280, 25-275, 30-270 and 25-265;

ii) SEQ ID NO: 4 or a fragment thereof selected from SEQ ID NO:4 positions 1-265, 1-270, 1-280, 1-290, 10-315, 15-315, 20-315, 25-315, 35-315, 10-290, 15-285, 20-280, 25-275, 30-270 and 25-265.

The invention includes sequences at least 85% identical (preferably 90%, more preferably 95%, and especially 99% identical) to the sequences of the invention as determined by the Smith-waterman algorithm.

In another of the present invention we provide an isolated and purified polynucleotide molecule comprising a nucleic acid sequence which encodes a polypeptide having at least about 90% homology to a member selected from (SEQ ID NO:2, SEQ ID NO:2 positions 1-160, SEQ ID NO:2 positions 150-315, and SEQ ID NO:2 positions 80-240). Isolated and purified polynucleotides of the present invention include sequences which comprise the mouse E2 cDNA sequence set out in SEQ ID NO: 1.

In a further aspect of the present invention we provide an isolated and purified polynucleotide molecule comprising a nucleic acid sequence which encodes a polypeptide having at least about 90% homology to a member selected from (SEQ ID NO:4, SEQ ID NO:4 positions 1-160, SEQ ID NO:4 positions 150-315, and SEQ ID NO:4 positions 80-240). Isolated and purified polynucleotides of the present invention include sequences which comprise the human E2 cDNA sequence set out in SEQ ID NO:3.

In a further aspect of the invention we provide fragments of the isolated and purified polynucleotide molecules of the present invention. By fragments we mean contiguous regions of the polynucleotide molecule including complementary DNA and RNA sequences, starting with short sequences useful as probes or primers of say about 8-50 bases, such as 10-30 bases or 15-35 bases, to longer sequences of up to 50, 100, 200, 500 or 1000 bases. Preferred fragments are at least 20 bases in length. Indeed any convenient fragment of the polynucleotide molecule may be a useful fragment for further research, therapeutic or diagnostic purposes. Further convenient fragments include those whose terminii are defined by restriction sites within the molecule of one or more kinds, such as any combination of Rsa1, Alu1 and Hinf1.

In a further aspect we provide homologues and orthologues of the isolated and purified polynucleotide molecules of the present invention. Preferred homologues and orthologues are polynucleotide molecules which display greater than 80% sequence homology, conveniently greater than 85%, for example 90%, to the E2 cDNA sequences set out in SEQ ID NO:1 and SEQ ID NO:3. A homologue may be a polynucleotide molecule from the same species i.e. a homologous family member, alternatively, the homologue may be a similar polynucleotide molecule from a different species such as human, useful in developing new therapies for the treatment of IRS and other related disorders such as NIDDM, obesity and atherosclerosis. By the term orthologue we mean a functionally equivalent molecule in another species. The full sequences of the individual homologues and orthologues may be determined using conventional techniques such as hybridisation, PCR and sequencing techniques, starting with any convenient part of the sequence set out in SEQ ID NO: 1 or SEQ ID NO:3.

In a further aspect of the invention we provide isolated and purified polynucleotide molecules capable of specifically hybridising to the polynucleotide molecules of the present invention. By specifically hybridising we mean that the polynucleotide hybridises by base-pair interactions, under stringent conditions, to the polynucleotide molecules of the present invention or to the corresponding complementary sequences. Experimental procedures for hybridisation under stringent conditions are well known to persons skilled in the art. For example, hybridisation filters may be incubated overnight at 42° C. in a solution comprising 50% formamide, 5×SSC (150 mM NaCl, 15 mM trisodium citrate), 50 mM sodium phosphate (pH 7.6) 5× Denhardt's solution, 10% dextran sulphate, and 20 μg/ml denatured salmon sperm DNA; followed by washing the filters in 0.1×SSC at about 65° C. Hybridisation techniques are thoroughly described in Sambrook J., Fritsch E. F. and Maniatis T., Molecular Cloning a Laboratory Manual, Cold Spring Harbor Laboratory Press, 1989.

In a further aspect we provide an expression vector comprising a polynucleotide molecule of the present invention.

A variety of mammalian expression vectors may be used to express the recombinant polypeptides of the present invention. Commercially available mammalian expression vectors which are suitable for recombinant expression include, pcDNA3 (Invitrogen), pMC1neo (Stratagene), pXT1 (Stratagene), pSG5 (Stratagene), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146), pUCTag (ATCC 37460), 1ZD35 (ATCC 37565), pLXIN, pSIR (CLONTECH), and pIRES-EGFP (CLONTECH).

Baculoviral expression systems may also be used with the present invention to produce high yields of biologically active polypeptides. Preferred vectors include the CLONTECH, BacPak™ Baculovirus expression system and protocols which are commercially available (CLONTECH, Palo Alto, Calif.).

Further preferred vectors include vectors for use with the mouse erythroleukaemia cell (MEL cell) expression system comprising the human beta globin gene locus control region (Davies et al., J. of Pharmacol. and Toxicol. Methods 33, 153-158).

Vectors comprising one or more polynucleotide molecules of the present invention may then be purified and introduced into appropriate host cells. Therefore in a further aspect we provide a transformed host cell comprising a polynucleotide molecule of the present invention.

The polypeptides of the present invention may be expressed in a variety of hosts such as bacteria, plant cells, insect cells, fungal cells and human and animal cells. Eukaryotic recombinant host cells are especially preferred. Examples include yeast, mammalian cells including cell lines of human, bovine, porcine, monkey and rodent origin, and insect cells including Drosophila and silkworm derived cell lines. Cell lines derived from mammalian species which may be used and which are commercially available include, L cells L-M(TK-) (ATCC CCL 1.3), L cells L-M (ATCC CCL 1.2), HEK 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26) and MRC-5 (ATCC CCL 171).

The expression vector may be introduced into host cells to express a polypeptide of the present invention via any one of a number of techniques including calcium phosphate transformation, DEAE-dextran transformation, cationic lipid mediated lipofection, electroporation or infection

The transformed host cells are propagated and cloned, for example by limiting dilution, and analysed to determine the expression level of recombinant polypeptide. Identification of transformed host cells which express a polypeptide of the present invention may be achieved by several means including immunological reactivity with antibodies described herein and/or the detection of biological activity.

In one embodiment of the invention, the E2 polypeptide can be present in the form of a transgenic non-mammal, such as a mouse. Therefore in a further aspect we provide a transgenic non-human mammal comprising a polynucleotide molecule of the present invention.

Transgenic non-human mammals are contemplated in which the gene of interest, preferably cut out from a vector and preferably in association with a promoter is introduced into the pronucleus of a mammalian zygote (usually by microinjection into one of the two nuclei (usually the male nucleus) in the pronucleus) and thereafter implanted into a foster mother. Preferably the transgenic non-human mammal is a mouse. A proportion of the animals produced by the foster mother will carry and express the introduced gene which has integrated into a chromosome. Usually the integrated gene is passed on to offspring by conventional breeding thus allowing ready expansion of stock. The reader is directed to the following publications: Simons et al. (1988), Bio/Technology 6:179-183; Wright et al. (1991) Bio/Technology 9:830-834; U.S. Pat. No. 4,873,191 and; U.S. Pat. No. 5,322,775. Manipulation of mouse embryos is described in Hogan et al, “Manipulating the Mouse Embryo; A Laboratory Manual”, Cold Spring Harbor Laboratory 1986.

If desired, host genes can be inactivated or modified using standard procedures as outlined briefly below and as described for example in “Gene Targeting; A Practical Approach”, IRL Press 1993. The target gene or portion of it is preferably cloned into a vector with a selection marker (such as Neo) inserted into the gene to disrupt its function. The vector is linearised then transformed (usually by electroporation) into embryonic stem (ES) cells (eg derived from a 129/O1a strain of mouse) and thereafter homologous recombination events take place in a proportion of the stem cells. The stem cells containing the gene disruption are expanded and injected into ablastocyst (such as for example from a C57BL/6J mouse) and implanted into a foster mother for development. Chimaeric offspring can be identified by coat colour markers. Chimeras are bred to ascertain the contribution of the ES cells to the germ line by mating to mice with genetic markers which allow a distinction to be made between ES derived and host blastocyst derived gametes. Half of the ES cell derived gametes will carry the gene modification. Offspring are screened (eg by Southern blotting) to identify those with a gene disruption (about 50% of progeny). These selected offspring will be heterozygous and therefore can be bred with another heterozygote and homozygous offspring selected thereafter (about 25% of progeny). Transgenic animals with a gene knockout can be crossed with transgenic animals produced by known techniques such as microinjection of DNA into pronuclei, sphaeroplast fusion (Jakobovits et al. (1993) Nature 362:255-258) or lipid mediated transfection (Lamb et al. (1993) Nature Genetics 5 22-29) of ES cells to yield transgenic animals with an endogenous gene knockout and foreign gene replacement.

ES cells containing a targeted gene disruption can be further modified by transforming with the target gene sequence containing a specific alteration, which is preferably cloned into a vector and linearised prior to transformation. Following homologous recombination the altered gene is introduced into the genome. These embryonic stem cells can subsequently be used to create transgenics as described above.

Polypeptides of the present invention may be expressed as fusion proteins, for example with one or more additional polypeptide domains added to facilitate protein purification. Examples of such additional polypeptides include metal chelating peptides such as histidine-typtophan modules that allow purification on immobilised metals (Porath, J., Protein Exp. Purif. 3:263 (1992)), protein A domains that allow purification on immobilised immunoglobulin, and the domain utilised in the FLAGS extension/affinity purification system (Immunex Corp, Seattle Wash.). The inclusion of cleavable linker sequences such as Factor XA or enterokinase (Invitrogen, San Diego Calif.) between the purification domain and the coding region is useful to facilitate purification. A preferred protein purification system is the CLONTECH, TALON™ nondenaturing protein purification kit for purifying 6×His-tagged proteins under native conditions (CLONTECH, Palo Alto, Calif.).

Therefore in a further aspect we provide a method for producing a polypeptide of the present invention, which method comprises culturing a transformed host cell comprising a polynucleotide of the present invention under conditions suitable for the expression of said polypeptide.

In another aspect of the present invention we provide an isolated or purified polypeptide which encodes an E2 polypeptide or a polypeptide fragment thereof of greater than 204 amino acids. By the term “isolated”, we mean that the polypeptide has been separated from those constituents that are normally present with it in nature. Preferably the E2 polypeptide or fragment thereof is selected from:

i) SEQ ID NO: 2 or a fragment thereof selected from SEQ D NO:2 positions 1-265, 1-270, 1-280, 1-290, 10-315, 15-315, 20-315, 25-315, 35-315, 10-290, 15-285, 20-280, 25-275, 30-270 and 25-265;

ii) SEQ ID NO: 4 or a fragment thereof selected from SEQ ID NO:4 positions 1-265, 1-270, 1-280, 1-290, 10-315, 15-315, 20-315, 25-315, 35-315, 10-290, 15-285, 20-280, 25-275, 30-270 and 25-265;

or a sequence at least 95% identical to any of these sequences.

In a further aspect of the present invention we provide a purified polypeptide comprising the mouse E2 amino acid sequence set out in SEQ ID NO.2 or a variant of SEQ ID NO.2 having at least about 90% homology to a member selected from (SEQ ID NO.2 positions 1-160, SEQ ID NO.2 positions 150-315, SEQ ID NO.2 positions 80-240), or a biologically active fragment thereof.

In a further aspect of the present invention we provide a purified polypeptide comprising the human E2 amino acid sequence set out in SEQ ID NO.4 or a variant of SEQ ID NO.4 having at least about 90% homology to a member selected from (SEQ ID NO.4 positions 1-160, SEQ ID NO.4 positions 150-315, SEQ ID NO.4 positions 80-240), or a biologically active fragment thereof.

A variant is a polynucleotide or polypeptide which differs from a reference polynucleotide or polypeptide, but which retains some of its essential characteristics. For example, a variant of an E2 polypeptide may have an amino acid sequence that is different by one or more amino acid substitutions, deletions and/or additions. The variant may have conservative changes (amino acid similarity), wherein a substituted amino acid has similar structural or chemical properties, for example, the replacement of leucine with isoleucine. Alternatively, a variant may have nonconservative changes, e.g., replacement of a glycine with a tryptophan. Guidance in determining which and how many amino acid residues may be substituted, inserted or deleted and the effect this will have on biological activity may be reasonably inferred from the present disclosure by a person skilled in the art and may further be found using computer programs well known in the art, for example, DNAStar software.

Amino acid substitutions may be made, for instance, on the basis of similarity in polarity, charge, solubility, hydrophobicity, hydrophilicity, and/or the amphipathic nature of the residues. Negatively charged amino acids, for example, include aspartic acid and glutamic acid; positively charged amino acids include lysine and arginine; and amino acids with uncharged polar head groups having similar hydrophilicity values include leucine, isoleucine, valine, glycine, alanine; asparagine, glutamine; serine, threonine, phenylalanine, and tyrosine.

Suitable substitutions of amino acids include the use of a chemically derivatised residue in place of a non-derivatised residue. D-isomers and other known derivatives may also be substituted for the naturally occurring amino acids. See, e.g., U.S. Pat. No. 5,652,369, Amino Acid Derivatives, issued Jul. 29, 1997. Example substitutions are set forth in Table 1.

“Homology” as used in this description is a measure of the similarity or identity of nucleotide sequences or amino acid sequences. In order to characterise the homology, subject sequences are aligned so that the highest order identity match is obtained. Identity can be calculated using published techniques. Computer program methods to determine identity between two sequences, for example, include DNAStar software (DNAStar Inc., Madison, Wis.); the GCG program package (Devereux, J., et al., Nucleic Acids Research 1984, 12(1):387); and BLASTP, BLASTN, FASTA (Atschul, S. F. et al., J Molec Biol 1990, 215:403). Homology as defined herein is determined conventionally using the well known computer program, BESTFIT (Wisconsin Sequence Analysis Package, Version 8 for Unix, Genetics Computer Group, University Research Park, 575 Science Drive, Madison, Wis., 53711). When using BESTFIT or another sequence alignment program to determine the similarity of a particular sequence to a reference sequence, the parameters are typically set such that the percentage identity is calculated over the full length of the reference nucleotide sequence or amino acid sequence and that gaps in homology of up to about 10% of the total number of nucleotides or amino acid residues in the reference sequence are allowed.

In a further aspect we provide polymorphic variants of the polynucleotides and polypeptides of the present invention. Polymorphisms are variations in polynucleotide or polypeptide sequences between one individual and another. DNA polymorphisms may lead to variations in amino acid sequence and consequently to altered protein structure and functional activity. Polymorphisms may also affect mRNA synthesis, maturation, transport and stability. Polymorphisms which do not result in amino acid changes (silent polymorphisms) or which do not alter any known consensus sequences may nevertheless have a biological effect, for example by altering mRNA folding or stability.

Knowledge of polymorphisms may be used to help identify patients most suited to therapy with particular pharmaceutical agents (this is often termed “pharmacogenetics”). Pharmacogenetics may also be used in pharmaceutical research to assist the drug selection process. Polymorphisms may be used in mapping the human genome and to elucidate the genetic component of diseases. The reader is directed to the following references for background details on pharmacogenetics and other uses of polymorphism detection: Linder et al. (1997), Clinical Chemistry, 43, 254; Marshall (1997), Nature Biotechnology, 15, 1249; International Patent Application WO 97/40462, Spectra Biomedical; and Schafer et al. (1998), Nature Biotechnology, 16, 33.

The polypeptides of the present invention may be genetically engineered in such a way that their interaction with other intracellular and membrane associated proteins are maintained but their effector function and biological activity are removed. A polypeptide genetically modified in this way is known as a dominant negative mutant. In the construction of a dominant negative mutant at least one amino acid residue position at a site required for activity in the native peptide is changed to produce a peptide which has reduced activity or which is devoid of detectable activity. Overexpression of the dominant negative mutant in an appropriate cell type down-regulates the effect of the endogenous polypeptide, thereby revealing the biological mechanisms involved in the control of metabolism.

Similarly, the polypeptides of the present invention may be genetically engineered in such a way that their effector function and biological activity are enhanced. The resultant overactive polypeptide is known as a dominant positive mutant. At least one amino acid residue position at a site required for activity in the native peptide is changed to produce a peptide which has enhanced activity. Overexpression of a dominant positive mutant in an appropriate cell type amplifies the response of the endogenous native polypeptide highlighting the regulatory mechanisms controlling cell metabolism.

Therefore in a further aspect we provide dominant negative and dominant positive mutants of the polypeptides of the present invention.

Novel sequences disclosed herein, may be used in another embodiment of the invention to regulate expression of E2 genes in cells by the use of antisense constructs. For example an antisense expression construct may be readily constructed using the pREP10 vector (Invitrogen Corporation). Transcripts are expected to modulate translation of the gene in cells transfected with the construct. Antisense transcripts are effective for modulating translation of the native gene transcript, and are capable of altering the effects (e.g., regulation of tissue physiology) herein described. Oligonucleotides which are complementary to and hybridisable with any portion of mRNA disclosed herein are contemplated for therapeutic use. U.S. Pat. No. 5,639,595, “Identification of Novel Drugs and Reagents”, issued Jun. 17, 1997, wherein methods of identifying oligonucleotide sequences that display in vivo activity are thoroughly described, is herein incorporated by reference. Antisense molecules may also be synthesised for use in antisense therapy, using techniques known to persons skilled in the art. These antisense molecules may be DNA, stable derivatives of DNA such as phosphorothioates or methylphosphonates, RNA, stable derivatives of RNA such as 2′-O-alkylRNA, or other oligonucleotide mimetics. U.S. Pat. No. 5,652,355, “Hybrid Oligonucleotide Phosphorothioates”, issued Jul. 29, 1997, and U.S. Pat. No. 5,652,356, “Inverted Chimeric and Hybrid Oligonucleotides”, issued Jul. 29, 1997, which describe the synthesis and effect of physiologically-stable antisense molecules, are incorporated by reference. Antisense molecules may be introduced into cells by microinjection, liposome encapsulation or by expression from vectors harboring the antisense sequence.

In a further aspect we provide an antibody specific for a polypeptide of the present invention.

Antibodies can be prepared using any suitable method, for example, purified polypeptide may be utilised to prepare specific antibodies. The term “antibodies” includes polyclonal antibodies, monoclonal antibodies, and the various types of antibody constructs such as for example F(ab′)₂, Fab and single chain Fv. Antibodies are defined to be specifically binding if they bind the antigen with a Kₐ of greater than or equal to about 10⁷M⁻¹. Affinity of binding can be determined using conventional techniques, for example those described by Scatchard et al., Ann. N.Y. Acad. Sci., 51:660 (1949).

Polyclonal antibodies can be readily generated from a variety of sources, for example, horses, cows, goats, sheep, dogs, chickens, rabbits, mice or rats, using procedures that are well-known in the art. In general, antigen is administered to the host animal typically through parenteral injection. The immunogenicity of antigen may be enhanced through the use of an adjuvant, for example, Freund's complete or incomplete adjuvant. Following booster immunisations, small samples of serum are collected and tested for reactivity to antigen. Examples of various assays useful for such determination include those described in: Antibodies: A Laboratory Manual, Harlow and Lane (eds.), Cold Spring Harbor Laboratory Press, 1988; as well as procedures such as countercurrent immuno-electrophoresis (CIEP), radioimmunoassay, radioimmunoprecipitation, enzyme-linked immuno-sorbent assays (ELISA), dot blot assays, and sandwich assays, see U.S. Pat. Nos. 4,376,110 and 4,486,530.

Monoclonal antibodies may be readily prepared using well-known procedures, see for example, the procedures described in U.S. Pat. Nos. RE 32,011, 4,902,614, 4,543,439 and 4,411,993; Monoclonal Antibodies, Hybridomas: A New Dimension in Biological Analyses, Plenum Press, Kennett, McKeam, and Bechtol (eds.), (1980).

The monoclonal antibodies of the invention can be produced using alternative techniques, such as those described by Alting-Mees et al., “Monoclonal Antibody Expression Libraries: A Rapid Alternative to Hybridomas”, Strategies in Molecular Biology 3: 1-9 (1990) which is incorporated herein by reference. Similarly, binding partners can be constructed using recombinant DNA techniques to incorporate the variable regions of a gene that encodes a specific binding antibody. Such a technique is described in Larrick et al., Biotechnology, 7: 394 (1989).

Once isolated and purified, the antibodies may be used to detect the presence of antigen in a sample using established assay protocols.

In a further aspect of the invention we provide a method for identifying a chemical compound capable of modulating the activity of E2 which method comprises:

(i) contacting a chemical compound with an E2 polypeptide of the invention described herein or

(ii) a transgenic non-human mammal as described herein; and

(iii) measuring an effect of the chemical compound on the activity of the E2 polypeptide or the transgenic non-human mammal.

An example of such a chemical compound is an antibody against E2 polypeptide. In a further aspect of the invention we provide a method for identifying a therapeutic agent capable of modulating the activity of E2 for use in the regulation of metabolism, which method comprises:

(i) contacting a candidate compound modulator with an E2 polypeptide comprising either

(a) the amino acid sequence set out in SEQ ID NO.2 or a variant of SEQ ID NO.2 having at least about 90% homology to a member selected from (SEQ ID NO.2 positions 1-160, SEQ ID NO.2 positions 150-315, SEQ ID NO.2 positions 80-240) or a biologically active fragment thereof; or

(b) the amino acid sequence set out in SEQ ID NO.4 or a variant of SEQ ID NO.4 having at least about 90% homology to a member selected from (SEQ ID NO.4 positions 1-160, SEQ ID NO.4 positions 150-315, SEQ ID NO.4 positions 80-240) or abiologically active fragment thereof; and

(ii) measuring an effect of the candidate compound modulator on the activity of the E2 polypeptide.

Activity as used herein refers to the ability of the therapeutic agent to mediate cell processes related to insulin resistance syndrome and other related disorders such as non-insulin dependent diabetes mellitus, dyslipidemia, obesity and atherosclerosis.

Modulation of the activity of E2 comprises either stimulation or inhibition. Thus a therapeutic agent capable of modulating the activity of E2 is an agent that either stimulates or inhibits the activity of E2. The terms “modulator of E2 activity” and “E2 modulator” are also used herein to refer to an agent that either stimulates or inhibits the activity of E2. The therapeutic agents of the invention have utility in the regulation of metabolism; in particular in the control of insulin resistance syndrome and other related disorders such as non-insulin dependent diabetes mellitus, dyslipidemia, obesity and atherosclerosis.

In a further aspect of the invention we provide a screen for identifying compounds which modulate the activity of E2, the invention extends to such a screen and to the use of compounds obtainable therefrom to modulate the activity of E2 in vivo.

Potential therapeutic agents which may be tested in the screen include simple organic molecules, commonly known as “small molecules”, for example those having a molecular weight of less than 2000 Daltons. The screen may also be used to screen compound libraries such as peptide libraries, including synthetic peptide libraries and peptide phage libraries. Other suitable molecules include antibodies, nucleotide sequences and any other molecules which modulate the activity of E2.

Once an inhibitor or stimulator of E2 activity is identified then medicinal chemistry techniques can be applied to further refine its properties, for example to enhance efficacy and/or reduce side effects.

It will be appreciated that there are many screening procedures which may be employed to perform the present invention. Examples of suitable screening procedures which may be used to identify an E2 modulator for use in the regulation of metabolism include rapid filtration of equilibrium binding mixtures, enzyme linked immunosorbent assays (ELISA), radioimmunoassays (RIA) and fluorescence resonance energy transfer assays (FRET). For further information on FRET the reader is directed to International Patent Application WO 94/28166 (Zeneca). Methods to identify potential drug candidates have been reviewed by BevanP et al., 1995, TIBTECH 13 115.

A preferred method for identifying a compound capable of modulating the activity of E2 is a scintillation proximity assay (SPA). SPA involves the use of fluomicrospheres coated with acceptor molecules, such as receptors, to which a ligand will bind selectively in a reversible manner (N Bosworth & P Towers, Nature, 341, 167-168, 1989). The technique requires the use of a ligand labelled with an isotope that emits low energy radiation which is dissipated easily into an aqueous medium. At any point during an assay, bound labelled ligands will be in close proximity to the fluomicrospheres, allowing the emitted energy to activate the fluor and produce light. In contrast, the vast majority of unbound labelled ligands will be too far from the fluomicrospheres to enable the transfer of energy. Bound ligands produce light but free ligands do not, allowing the extent of ligand binding to be measured without the need to separate bound and free ligand.

Cellular assay systems may be used to further identify E2 modulators for use in the regulation of metabolism.

Therefore in a further aspect of the invention we provide a method for identifying a therapeutic agent capable of modulating the activity of E2 for use in the regulation of metabolism, which method comprises:

(i) contacting a candidate compound modulator with a host-cell which expresses an E2 polypeptide comprising either

(a) the amino acid sequence set out in SEQ ID NO.2 or a variant of SEQ ID NO.2 having at least about 90% homology to a member selected from (SEQ ID NO.2 positions 1-160, SEQ ID NO.2 positions 150-315, SEQ ID NO.2 positions 80-240) or a biologically active fragment thereof, or

(b) the amino acid sequence set out in SEQ ID NO.4 or a variant of SEQ ID NO.4 having at least about 90% homology to a member selected from (SEQ ID NO.4 positions 1-160, SEQ ID NO.4 positions 150-315, SEQ ID NO.4 positions 80-240) or a biologically active fragment thereof; and

(ii) measuring an effect of the candidate compound modulator on the activity of E2.

A preferred cellular assay system for use in the method of the invention is a two-hybrid assay system. The two-hybrid system utilises the ability of a pair of interacting proteins to bring the activation domain of a transcription factor into close proximity with its DNA-binding domain, restoring the functional activity of the transcription factor and inducing the expression of a reporter gene (S Fields & O Song, Nature, 340, 245-246, 1989). Commercially available systems such as the Clontech Matchmaker™ systems and protocols may be used with the present invention.

Other preferred cellular assay systems include measurement of changes in the levels of intracellular signalling molecules such as cyclic-AMP, intracellular calcium ions, or arachidonic acid metabolite release. These may all be measured using standard published procedures and commercially available reagents. In addition the polynucleotides of the present invention may be transfected into appropriate cell lines that have been transfected with a “reporter” gene such as bacterial lacZ, luciferase, aequorin or green fluorescent protein that will “report” these intracellular changes (Egerton et al, J. Mol, Endocrinol, 1995, 14(2), 179-189).

According to a further aspect of the invention we provide a method of making a pharmaceutical composition which comprises:

(i) a method for identifying a chemical compound capable of modulating the activity of E2; and

(ii) mixing the compound thus identified with a pharmaceutically acceptable diluent or carrier.

In a further aspect of the present invention we provide a novel E2 modulator, or a pharmaceutically acceptable salt thereof, for use in a method of treatment of metabolic diseases of the human or animal body by therapy.

Examples of metabolic diseases which may be treated using a compound of the invention include insulin resistance syndrome, non-insulin dependent diabetes mellitus, dyslipidemia, obesity and atherosclerosis.

According to a further aspect of the invention, we provide a pharmaceutical composition which comprises a novel E2 modulator, or a pharmaceutically acceptable salt thereof, in association with a pharmaceutically acceptable diluent or carrier.

The composition may be in the form suitable for oral use, for example a tablet, capsule, aqueous or oily solution, suspension or emulsion; for topical use, for example a cream, ointment, gel or an aqueous or oily solution or suspension; for nasal use, for example a snuff, nasal spray or nasal drops; for rectal use, for example a suppository; for administration by inhalation, for example as a finely divided powder such as a dry powder, a microcrystalline form or a liquid aerosol; for sub-lingual or buccal use, for example a tablet or capsule; or for parenteral use (including intravenous, subcutaneous, intramuscular, intravascular or infusion), for example a sterile aqueous or oily solution or suspension. In general, the above compositions may be prepared in a conventional manner using conventional excipients.

The invention also provides a method of treating a metabolic disease or medical condition mediated alone or in part by E2, which comprises administering to a warm-blooded animal requiring such treatment an effective amount of an E2 modulator as defined above.

The invention also provides the use of an E2 modulator in the production of a medicament for use in the treatment of a metabolic disease.

The amount of active ingredient that is combined with one or more excipients to produce a single dosage form will necessarily vary depending on the subject treated and the particular route of administration. For example, a formulation intended for oral administration to humans will generally contain for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient.

The size of the dose for therapeutic or prophylactic purposes of an E2 modulator will naturally vary according to the nature and severity of the immune disease, the age and sex of the patient, and the route of administration, according to well known principles of medicine.

In using an E2 modulator for therapeutic or prophylactic purposes it will generally be administered so that a daily dose in the range for example 0.5 mg to 75 mg per kg body weight is received, given if required in divided doses. In general lower doses will be administered when a parenteral route is employed. Thus for example, for intravenous administration, a dose in the range for example 0.5 mg to 30 mg per kg body weight will generally be used. Similarly, for administration by inhalation a dose in the range for example 0.5 mg to 25 mg per kg body weight will be used.

The invention will now be illustrated but not limited by reference to the following Tables, Examples and Figures. Unless indicated otherwise, the techniques used are those detailed in well known molecular biology textbooks such as Sambrook, Fritsch & Maniatis, Molecular Cloning a Laboratory Manual, second edition, 1989, Cold Spring Harbor Laboratory Press.

### FIGURE LEGENDS

FIG. 1 shows the full length mouse E2 cDNA (SEQ ID NO. 1)

FIG. 2 shows mouse E2 protein sequence (SEQ ID NO.2)

FIG. 3 shows human E2 cDNA (SEQ ID NO.3)

FIG. 4 shows human E2 protein sequence (SEQ ID NO.4)

FIG. 5 shows a sequence alignment of human E2 cDNA with EMBL AF 092133.

FIG. 6 shows a sequence alignment of human E2 protein with EMBL AF 092133.

FIG. 7 shows the relative expression levels of E2 in epididymal fat of animals treated with rosiglitazone, 30 μmol/kg/day daily. Study with 3 animals in each group. Real time PCR quantitation of E2 expression in epididymal fat was performed after 0, 1, 3 and 7 days.

FIG. 8 shows a comparison of E2 expression in mesenterial fat from lean mice, untreated ob/ob mice, and ob/ob mice treated with rosiglitazone for 7 days (5 animals in each group).

FIG. 9 shows the relative expression levels of E2 in different tissues isolated from lean and obese animals.

FIG. 10 shows a Northern blot analysis of E2 RNA from tissues isolated from lean mice, untreated ob/ob mice, and ob/ob mice treated with rosiglitazone for 7 days. (Control blot used ribosomal protein 36B4).

FIG. 11 shows a Western blot analysis of CHO cells expressing recombinant mouse E2. Lane 1. CHO cells with expression vector pcDNA3.1 comprising DNA encoding E2. Lane 2. CHO cells with pc DNA3.1 vector alone.

### TABLES

TABLE 1
Examples of conservative amino acid substitutions

| Original residue | Example conservative substitutions |
|---|---|
| Ala (A) | Gly; Ser; Val; Leu; Ile; Pro |
| Arg (R) | Lys; His; Gln; Asn |
| Asn (N) | Gln; His; Lys; Arg |
| Asp (D) | Glu |
| Cys (C) | Ser |
| Gln (Q) | Asn |
| Glu (B) | Asp |
| Gly (G) | Ala; Pro |
| His (H) | Asn; Gln; Arg; Lys |
| Ile (I) | Leu; Val; Met; Ala; Phe |
| Leu (L) | Ile; Val; Met; Ala; Phe |
| Lys (K) | Arg; Gln; His; Asn |
| Met (M) | Leu; Tyr; lle; Phe |
| Phe (F) | Met; Leu; Tyr; Val; Ile; Ala |
| Pro (P) | Ala; Gly |
| Ser (S) | Thr |
| Thr (T) | Ser |
| Trp (W) | Tyr; Phe |
| Tyr (Y) | Trp; Phe; Thr; Ser |
| Val (V) | Ile; Leu; Met; Phe; Ala |

TABLE 2

Primer sequences

| | |
|---|---|
| Primer | Sequence |

| | |
|---|---|
| H-T₁₁-A | AAGCTTTTTTTTTTTA |
| H-T₁₁-C | AAGCTTTTTTTTTTTC |
| H-T₁₁-G | AAGCTTTTTTTTTTTG |
| H-AP-1 | AAGCTTGATTGCC |
| H-AP-2 | AAGCTTCGACTGT |
| H-AP-3 | AAGCTTTGGTCAG |
| H-AP-4 | AAGCTTCTCAACG |
| H-AP-5 | AAGCTTAGTAGGC |
| H-AP-6 | AAGCTTGCACCAT |
| H-AP-7 | AAGCTTAACGAGG |
| H-AP-8 | AAGCTTTTACCGC |
| H-AP-9 | AAGCTTCATTCCG |
| H-AP-10 | AAGCTTCCACGTA |
| Rgh | GACGCGAACGAAGCAAC |
| Lgh | CGACAACACCGATAATC |

### EXAMPLES

### Example 1

Animals, Cell Culture and Treatments.

Nine weeks old ob/ob mice were treated for seven days with rosiglitazone at 30 μmol/kg/day. The drug was administered orally using a gavage. Control animals were fed vehicle (0.1% dimethylsulphoxide (DMSO)). To reduce variation in genetic background male sibling pairs were used with one being treated with drug and the other with vehicle. The animals had free access to water and normal mouse chow.

3T3-L1 cells were grown in 175 cm² flasks to confluency. Dexamethasone at 2 μg/ml and methylisobutyl-xanthine at 0.5 μM were then included in the cell culture medium. This treatment was continued for two weeks. This drives the differentiation of the cells to adipocytes. The dexamethasone and methylisobutyl-xanthine were removed and the cells were thereafter treated with rosiglitazone at 1 μM for 24 hours. Control cells were also treated with dexamethasone/methylisobutyl-xanthine but with vehicle instead of rosiglitazone.

### Example 2

Tissue Isolation and RNA Extraction.

Treated and control mice were killed and tissues were removed. Liver, mesenterial fat, epididimus fat, brown fat, white fibres from quadriceps (quadri/white), red fibres from quadriceps (quadri/red) and heart were isolated. Care was taken to remove contaminating tissues, blood and hair. All tissues were rapidly removed and snap-frozen in liquid nitrogen within 2 minutes after the animal was killed. Tissues were weighed and RNASTAT-60 (AMS Biotechnology) was added. Tissues were then homogenised with a Turrax-blender for one minute on ice.

Total RNA was extracted according to the suppliers protocol. Briefly, for tissue amounts up to 100 mg, 1 ml of extraction media was added and the tissue homogenised. The organic and water phases were separated by centrifugation. The upper, water phase was isolated and RNA precipitated with one volume of isopropanol. The RNA pellet was washed with ice-cold 75% ethanol. The RNA pellet was dried and dissolved in diethyl pyrocarbonate (DEPC) treated water.

For RNA extraction of 3T3-L1 cells the cell culture medium was poured off and RNASTAT-60 added. RNA was extracted as described above.

To remove residual DNA the total RNA preparation was treated with DNAse. 50 μg RNA was incubated at 37° C. with 5 U DNAse (RQ1 DNAse, Promega) in 10 mM CaCl₂; 6 mM MgCl₂; 10 mM NaCl and 40 mM Tris-HCl pH 7.9 in a final volume of 100 μl. After 15 minutes the reaction was stopped by adding 4 μl 0.5 M ethylenediamine tetra-acetic acid (EDTA).

Protein was removed with a phenol/chloroform/isoamylalcohol extraction. The RNA was ethanol precipitated, re-dissolved in DEPC treated water and quantified by spectrophotometry at 260 μm. The quality of the RNA was also checked on a 1% agarose gel.

### Example 3

Differential Display.

The differential display was performed using reagents from GeneHunter, alternative procedures familiar to a person skilled in the art are detailed in Sambrook, Fritsch & Maniatis (ibid).

In three parallel reaction conditions, total RNA was reverse transcribed using three different anchored primers, H-T₁₁-A, H-T₁₁-G and H-T₁₁-C (Table 2). Each reaction was performed in duplicate. 0.2 μg of total RNA in 13.4 μl water was mixed with 1.6 μl 250 μM deoxy nucleotide triphosphates (dNTP); 4 μl 5× reverse transcription buffer (125 mM Tris-HCl pH 8.3, 188 mM KCl, 7.5 mM MgCl₂, 25 mM dithiothreitol (DTT)) and 2 μl 2 μM anchored primer. Samples were incubated in a thermocycler, at 65° C. for 5 min., 37° C. for 60 min. and 75° C. for 5 min. After five minutes at 37° C. Moloney murine leukaemia virus (MMLV) reverse transcriptase was added.

Amplification of cDNA was carried out by polymerase chain reaction (PCR). Reactions were set up using combinations of the three anchored primers and ten different random primers (Table 2). For each primer combination the following reaction was set up: 9.2 μl water; 2 μl 10×PCR buffer (100 mM Tris-HCl pH 8.4, 500 mM KCl, 15 mM MgCl₂ and 0.01% gelatin); 1.6 μl 25 μM dNTP; 2 μl 2 μM anchored primer; 2 μl RT reaction mix; 0.25 μl α-[³³P]dATP, 2000 Ci/mmol and 0.2 μl AmpliTaq DNA polymerase (Perkin-Elmer). Samples were incubated in a thermocycler, using the following temperature cycle: (1) 94° C. for 30 sec.; (2) 40° C. for 2 min.; (3) 72° C. for 30 sec.; (4) repeat steps 1-3 for 40 cycles; (5) 72° C. for 5 min.

After the amplification 3.5 μl of the PCR reaction were mixed with 2 μl loading dye (95% formamide, 10 mM EDTA pH 8.0, 0.09% Xylene cyanole FF and 0.09% bromophenol blue). Immediately before loading on 6% polyacrylamide gel with urea, the samples were denatured at 80° C. PCRs with the same primer combination from treated and untreated tissues or cells were loaded side by side.

When the slower migrating xylene dye reached the bottom of the gel the electrophoresis was stopped. The gel was transferred to a filter paper and dried in a vacuum gel dryer. Radioactivity was detected by placing the dried gel against Hyperfilm MX™ (Amersham). Overnight exposures were generally sufficient.

Bands which were differentially expressed i.e. appeared in duplicate samples from either the treated or untreated tissue but not both; were isolated by cutting out the band from the dried gel with a scalpel. To make sure that the correct band was cut out a second autoradiography film was exposed to the dried gel.

Isolated bands were boiled in 100 μl water for 15 min. The gel and filter paper were spun down and the supernatant transferred to a fresh tube. The isolated DNA fragments were re-amplified using the same PCR protocol as described above with one change: the dNTP concentration in the re-amplification was 20 μM. PCR products were then analysed on 1% agarose.

If the amplification gave a PCR product of expected size the PCR reaction mixture was used for ligation of the PCR product into pCRTRAP vector (GeneHunter). Five μl water was mixed with 2 μl linearised pCRTRAP; 1 μl 10× ligation buffer (GeneHunter or Sambrook et al., ibid); 2.5 μl PCR product and 0.5 μl T4 DNA ligase (100 U). The ligation reaction was incubated at 16° C. overnight.

Ten μl of ligation reaction mixture were transformed into 100 μl GH-competent cells (GeneHunter or Sambrook et al., ibid). Bacteria were plated onto LB agar plates with tetracycline (20 μg/ml). Colonies which appeared after overnight incubation at 37° C. were collected and lysed at 95° C. for 10 min. in 50 μl lysis buffer (GeneHunter or Sambrook et al., ibid).

The size of the insert was checked using PCR with a vector specific primer pair: Rgh, Lgh (Table 2). 10.2 μl water were mixed with 2 μl 10×PCR buffer; 1.6 μl 250 μM dNTP; 2 μl 2 μM Lgh primer; 2 μl 2 μM Rgh primer; 0.2 μl AmpliTaq DNA polymerase (1 U) and 2 μl colony lysate. PCR rections were performed at (1) 94° C. for 30 sec., (2) 52° C. for 40 sec., (3) 72° C. for 1 min., (4) repeat steps 1-3 for 40 cycles, (5) 72° C. for 5 min. PCR products were analysed on 1.5% agarose.

In general five positive colonies were used to inoculate 5 ml LB medium with tetracycline. Cultures were incubated over night. Bacterial cells were spun down and the pellet used for a Wizard (Promega) plasmid DNA miniprep.

### Example 4

DNA Sequencing

Inserts were sequenced using the Rgh or Lgh primer with the Thermocycler kit for dye terminator cycle sequencing (Perkin Elmer). Alternative procedures familiar to the person skilled in the art are detailed in Sambrook et al., ibid.

### Example 5

Bioinformatic Analysis.

Sequences originating from one differential display band were compared using sequence analysis software Lasergene/Seqman, DNA-star. Consensus sequence was used in the bioinformatic analysis. The vector sequence was trimmed away using Lasergene/Megalign. Resulting insert sequence was run against several sequence databases, EMBL non-EST, EMBL EST, GeneseqN using blastn (Basic Local Alignment Search Tool, Karlin S., and Altschul S. F., 1993, Proc. Natl. Acad. Sci. USA, 90, 5873-5877). Mouse ESTs were checked against Unigene/mouse.

### Example 6

Results of Differential Display.

In the reverse transcription step three different anchored primers were used in three independent reaction conditions. These primers have a poly T portion which will hybridise to the poly A tail in the 3′ end of mRNA. The last base is either C, G or A. This procedure subdivides all poly A transcripts into three cDNA pools.

For each of the cDNA pools PCR reactions were set up using ten different random primers (Table 2). This procedure subdivides and amplifies the cDNA pools further. The primer combinations used in this study typically generated approximately 150 fragments/primer combination.

Using three different anchored and ten different random primers a total of 4500 fragments were generated for each tissue. It is estimated that 15000 genes are expressed at any given moment in a cell (Axel R., et al., Cell 1976, 7(2), 247-254). Using this estimation and assuming that each gene will generate only one fragment, it can be calculated that 1 in 3 of the expressed genes in each tissue has been analysed.

Of the analysed fragments approximately 150 were detected and isolated as being differentially expressed. In the individual tissues between 7 to 23 differentially expressed fragments were detected with a mean around 15. This indicates that approximately 0.1% of expressed genes were affected by the drug treatment. The highest number of differentially expressed fragments was found in brown adipose tissue, followed by liver, epididimus fat, mesenterial fat, quadri/white, quadri/red and heart. 3T3-L1 cells were affected to the same extent as liver tissue. This indicates that of the tissues studied here brown fat was the most affected by the drug treatment while heart was the least affected. Liver and fat tissues were more affected than muscle tissues.

### Example 7

Fragments, Up or Down Regulated by Rosiglitazone Treatment, Found in Several Tissues.

Following rosiglitazone treatment the levels of expression of two thirds of the fragments were up-regulated and the remaining one third were down-regulated. In all tissues studied both up- and down-regulated gene expression was observed. The highest relative up-regulation of expression was detected in brown fat while the highest relative down-regulation was detected in heart. Using Lasergene/Seqman software all fragments in this study were compared to each other. In seven cases a fragment was found to be differentially expressed in two tissues. In four of these cases the fragment was similarly regulated in the two tissues. In two cases the fragment was differently regulated in the two tissues. A fragment of stearoyl-CoA desaturase was up-regulated in mesenterial fat and brown fat, whereas another fragment of stearoyl-CoA desaturase, which was also found in brown fat, was down-regulated by rosiglitazone treatment.

### Example 8

Bioinformatics Analysis.

The sequences obtained from the differential display experiment were compared to sequences found in public DNA databases. EMBL non-EST and EMBL EST were searched using the blastn algorithm. Hits with P(N) values lower than 10⁻¹⁰ in the EMBL non-EST database were used to identify fragments. Hits from mammals other than mouse were used for identification only if the differential display fragment aligned to the coding part of the non-mouse cDNA or gene. If no hits were obtained in the EMBL non-EST database the EMBL EST database was searched. Only hits from mouse with P(N) lower than 10⁻¹⁰ were recorded. If no significant non-EST or EST entries were found in any of the databases the differential display fragment was designated “unknown”. Somewhat less than half of the fragments in this study returned known genes when analysed against sequence databases. One quarter was only identified as EST and one quarter as unknown. In all tissues and cells studied approximately the same proportion of known, EST and unknown sequences were observed.

### Example 9

Unigene Cluster, Mapped, Mutants.

The accession number for the EST with the lowest P(N) value was used to search the Unigene/mouse database. This database contains clusters of ESTs together with information about tissue distribution and, in some cases, mapping information. Of all differential display fragments which were only identified as ESTs about half were found in Unigene. Of these Unigene clusters one third was mapped. The mapping information can be used to search the Mouse Genome Database, Jackson Laboratories, to find phenotypes in this genetic region.

### Example 10

Differential Expression of Mouse E2 cDNA

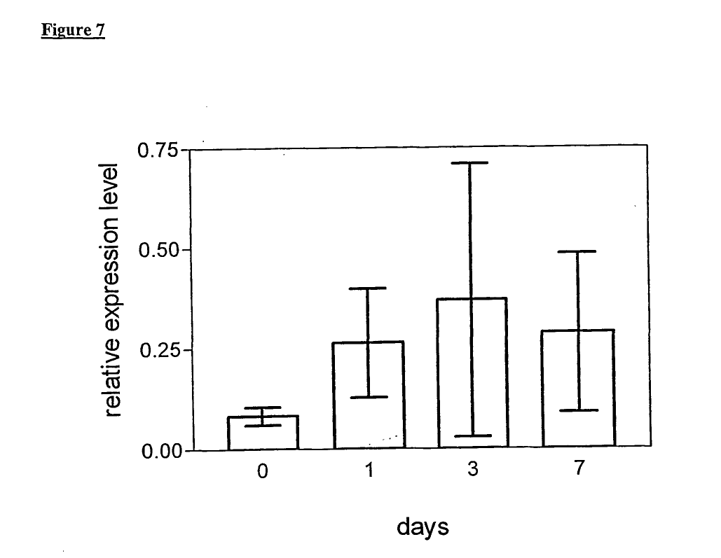

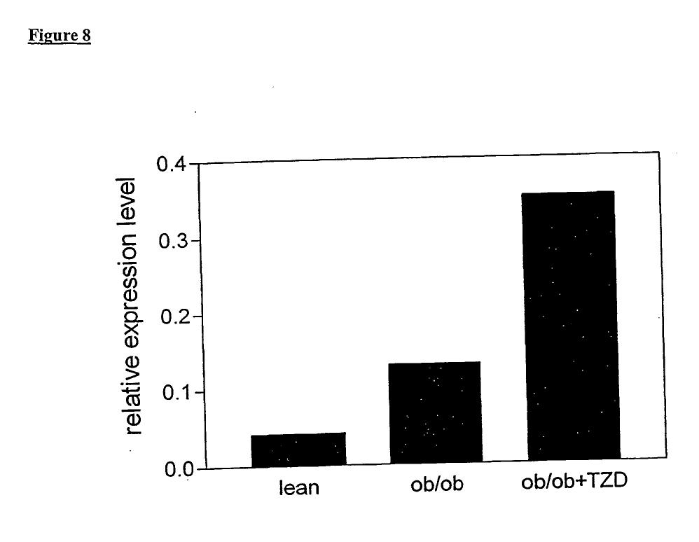

One of the differentially expressed sequences was mouse E2 mRNA. The E2 message was up-regulated after 7 days of rosiglitazone treatment (administered daily, 30 μmol/kg/day) in epididymal fat tissue (FIG. 7). Real time PCR quantitation on tissues from another set of identically treated animals showed that the up-regulation occurred also in mesenterial fat tissue (FIG. 8). The measurements were done on pooled cDNA from 5 animals, hence the lack of error bars.

In addition, FIG. 8 clearly shows that the E2 mRNA levels are significantly elevated in obese (ob/ob) compared with lean (−/ob) animals. Treatment with rosiglitazone enhances this up-regulation.

FIG. 7 shows that the expression levels increase substantially after the first rosiglitazone administration to ob/ob mice, and is further increased over a 7 day period. The up-regulation of E2 precedes the effects on plasma glucose and triglycerides which are not lowered by the first administration of rosiglitazone.

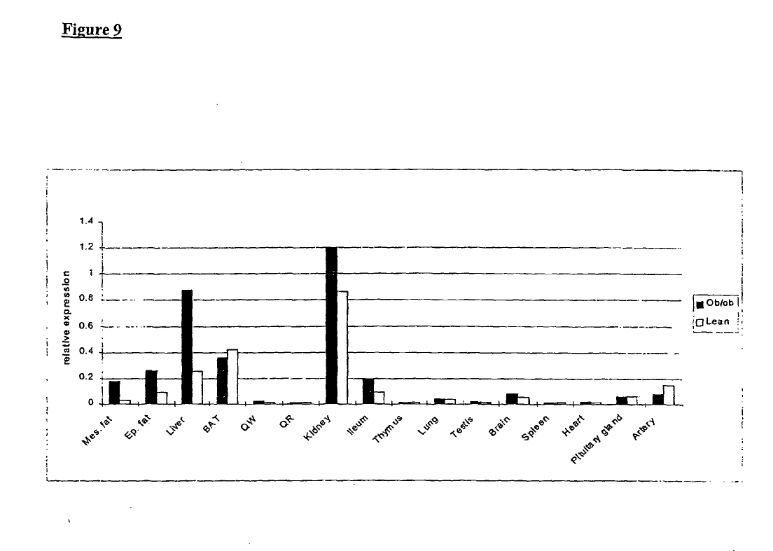

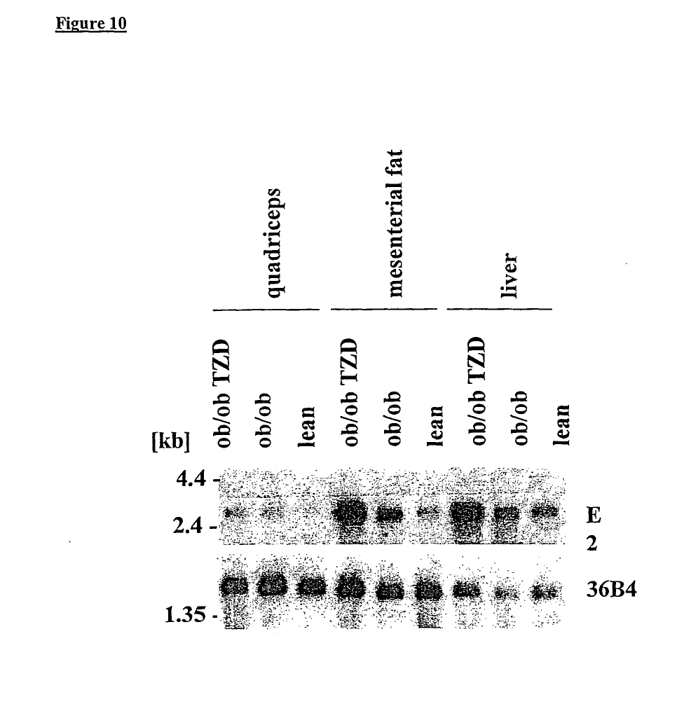

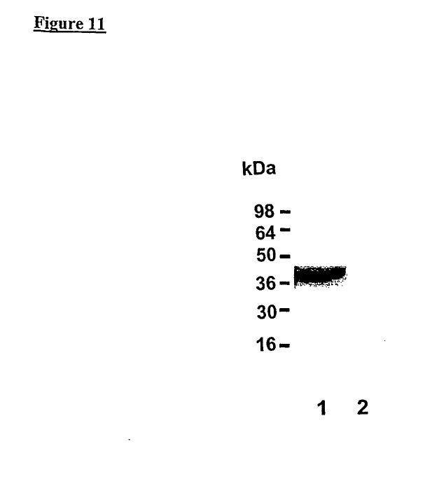

The tissue distribution of E2 transcripts in mouse and human tissues were analysed using real time quantitative PCR and Northern blot detection. FIG. 9 shows the expression levels in various tissues from lean and obese animals. The expression was found to be up-regulated in several tissues in obese animals. Besides epididymal and mesenterial fat, the up-regulation is most pronounced in liver and kidney. Northern blots with RNA from various tissues showed a transcript with clear up-regulation in obese animals and after rosiglitazone treatment (FIG. 10).

### Example 11

Cloning of Mouse E2 cDNA

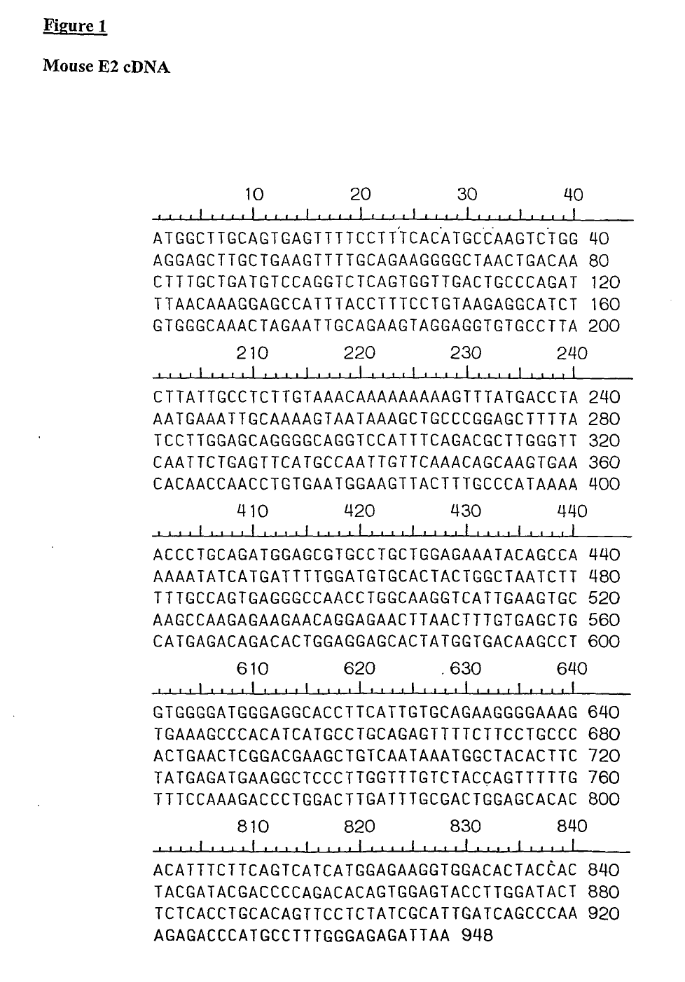

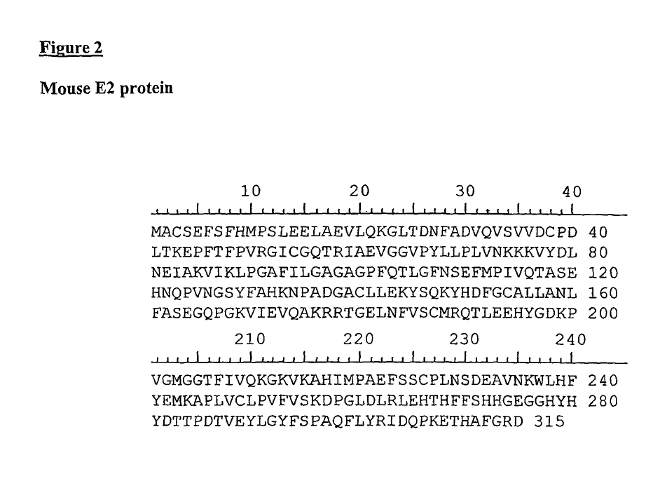

The complete mouse E2 cDNA was cloned from RNA purified from fat tissue by RACE using the Marathon cDNA Amplification Kit according to the manufacturers instructions (Clontech). The DNA sequence encoding mouse E2 is shown in FIG. 1 (SEQ ID NO: 1) and the translated protein in FIG. 2 (SEQ ID NO:2).

### Example 12

Cloning of Human E2 cDNA

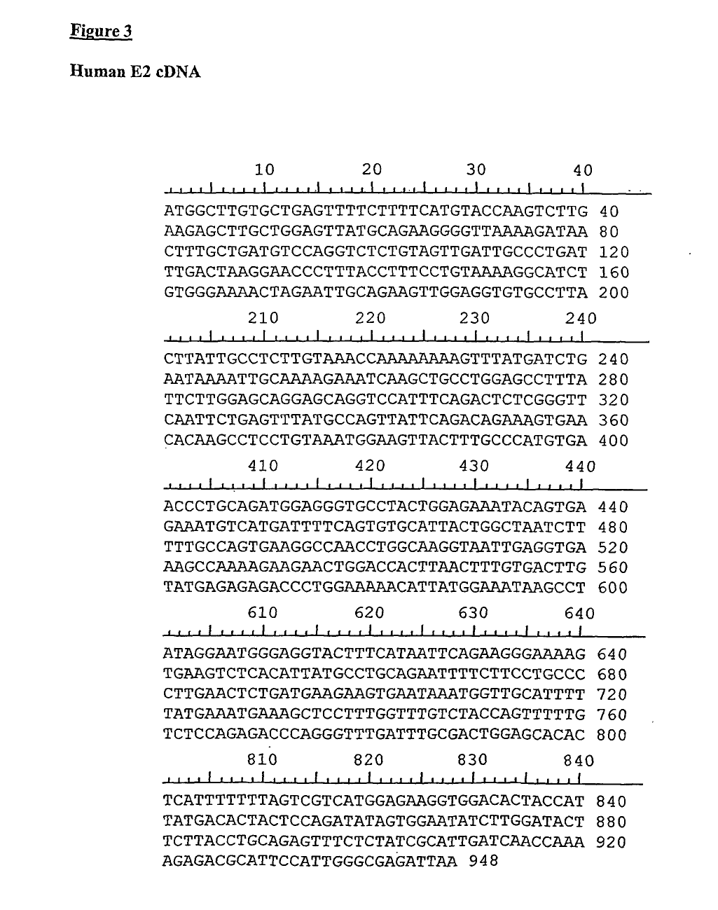

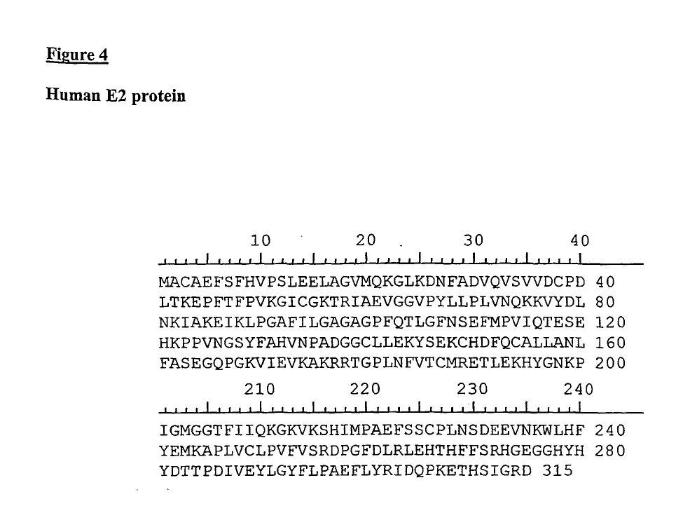

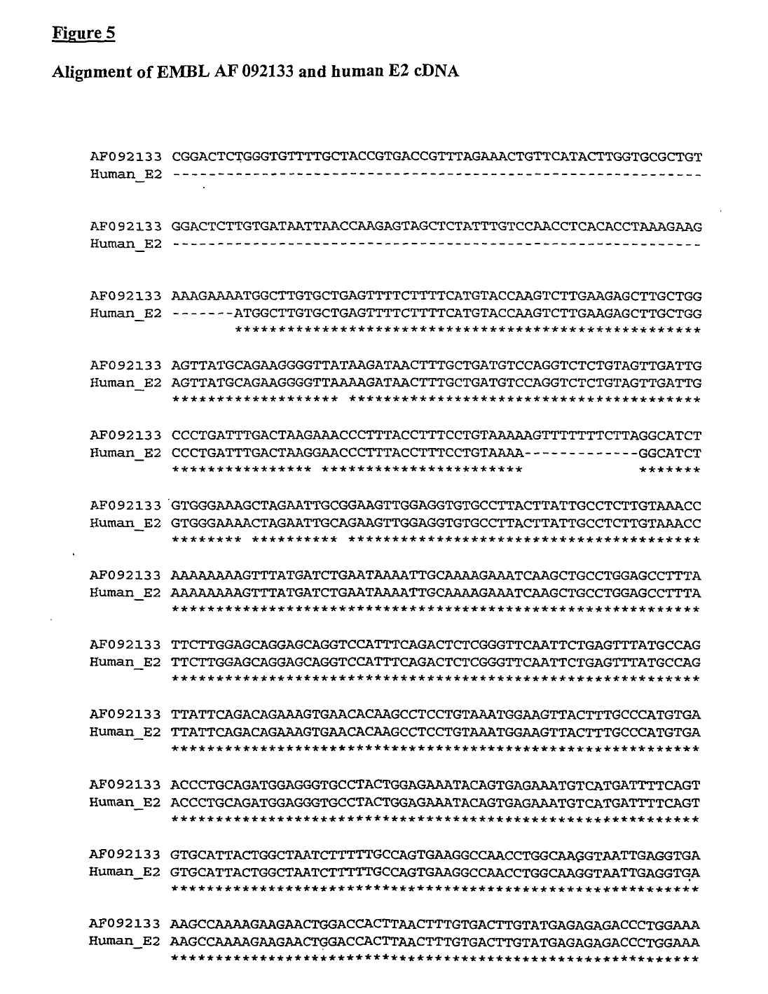

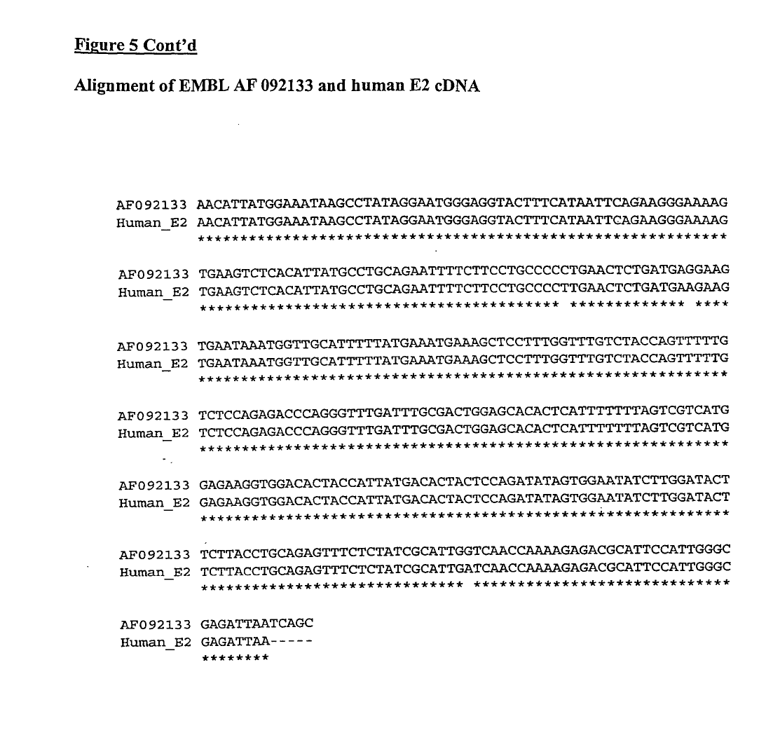

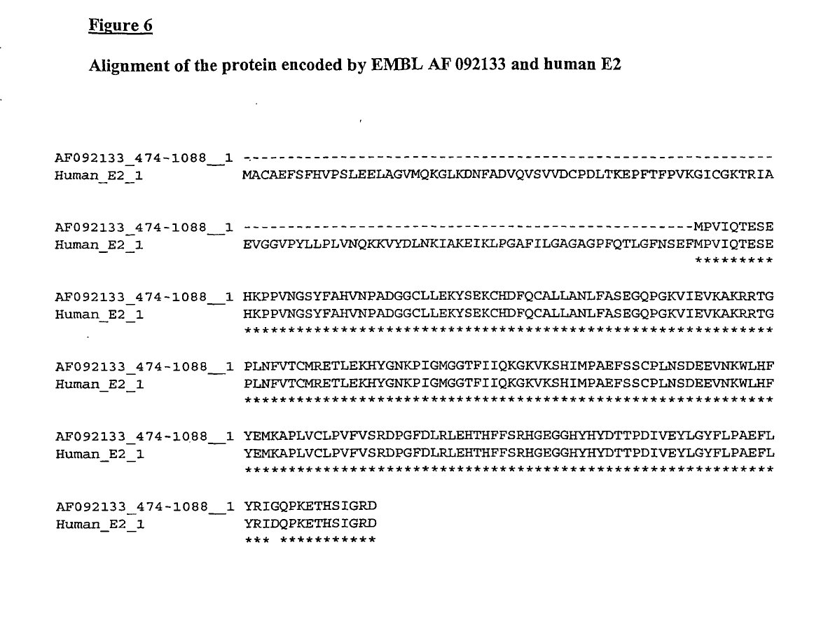

Homology search of the EMBL database with the obtained mouse E2 cDNA sequence revealed a sequence, EMBL AF092133, with 85% homology to the mouse E2 cDNA. It was assumed that this represented a possible human E2 analogue. PCR primers were designed based on EMBL AF092133 and the human E2 cDNA was cloned by PCR from human kidney RNA. The complete sequences were assembled using the Lasergene Seqman program (DNASTAR inc.). The DNA sequence encoding human E2 is shown in FIG. 3 (SEQ ID NO:3). SEQ ID NO:3 differs from EMBL AF092133 by a 13 base pair insertion at position 280 in EMBL AF092133 leading to a frame shift compared to SEQ ID NO:3. EMBL AF092133 codes for a 204 amino acid protein. Human E2 comprises 315 amino acids, shown in FIG. 4 (SEQ ID NO:4). EMBL AF092133 lacks 111 amino acids in the N-terminal compared to SEQ ID NO:4.

### Example 13

Expression of Recombinant Mouse E2 cDNA

DNA encoding mouse E2 was ligated into the expression vector pcDNA3.1. CHO cells were transfected with 6 □g of E2 plasmid DNA by Lipofectamine-mediated transfection. Cells were grown, harvested and resolved on 4-20% gradient SDS gel and transformed to nitrocellulose membrane. E2 protein was identified by affinity-purified antibodies directed against a peptide epitope on E2 and detected by ECL.

### Example 14

Generation of Transgenic Mice.

DNA constructs containing the mouse E2 cDNA under the control of the mouse metallothionein I (Mt-1) promoter (EMBL accession no. M11534) and regulatory parts of the human growth hormone (GH) gene (EMBL accession no. M13438, nucleotides 496-2641 where the ATG at nucleotide position 559 was mutated and a Not-1 site was introduced at position 913) were generated by subcloning the cDNA into the Not I site in the modified human GH gene.

Transgenic mice were generated in c57BL/6JxCBA-f2 embryos. Identification of transgenic animals was made by PCR using DNA extracted from tail sections obtained at 2 weeks of age. All transgenic animals included were heterozygous for the transgene, animals result from mating of transgenic males to wildtype females. Non-transgenic littermates were used as controls.

Founders were obtained and E2 mRNA expression was analysed in 3 lines using real time PCR. The transgene was expressed in liver and adipose tissue (only tissues examined). Different levels of overexpression of the transgene was obtained in respective line. Lines with high expression in adipose tissue is being characterised in more detail and will be used as models to study the function of E2 and the effects of elevated levels of expression of E2. Transgenic animals overexpressing E2 can also be used in assay and screens to identify and evaluate the effect of compounds able to modulate the activity or amount of E2.

1 An isolated polynucleotide molecule comprising a nucleic acid sequence which encodes an E2 polypeptide or a polypeptide fragment thereof of greater than 204 amino acids.

2 A polynucleotide according to claim 1 wherein the E2 polypeptide or fragment thereof is selected from:
i) SEQ ID NO: 2 or a fragment thereof selected from SEQ ID NO:2 positions 1-265, 1-270, 1-280, 1-290, 10-315, 15-315, 20-315, 25-315, 35-315, 10-290, 15-285, 20-280, 25-275, 30-270 and 25-265;
ii) SEQ ID NO: 4 or a fragment thereof selected from SEQ ID NO:4 positions 1-265, 1-270, 1-280, 1-290, 10-315, 15-315, 20-315, 25-315, 35-315, 10-290, 15-285, 20-280, 25-275, 30-270 and 25-265
or a sequence at least 95% identical to any of these sequences.

3 A polynucleotide according to claim 1 wherein the E2 polypeptide is selected from SEQ ID NO:2 or SEQ ID NO:4.

4 A polynucleotide according to claim 1 selected from SEQ ID NO 1 or SEQ ID NO 3.

5 An expression vector comprising a polynucleotide molecule defined in any of claims 1-4.

6 A transformed host cell or a transgenic non-human mammal comprising a polynucleotide molecule defined in any one of claims 1-4.

7 An isolated polypeptide which encodes an E2 polypeptide or a polypeptide fragment thereof of greater than 204 amino acids.

8 An E2 polypeptide or fragment thereof according to claim 7 selected from:
i) SEQ ID NO: 2 or a fragment thereof selected from SEQ ID NO:2 positions 1-265, 1-270, 1-280, 1-290, 10-315, 15-315, 20-315, 25-315, 35-315, 10-290, 15-285, 20-280, 25-275, 30-270 and 25-265;
ii) SEQ ID NO: 4 or a fragment thereof selected from SEQ ID NO:4 positions 1-265, 1-270, 1-280, 1-290, 10-315, 15-315, 20-315, 25-315, 35-315, 10-290, 15-285, 20-280, 25-275, 30-270 and 25-265
or a sequence at least 95% identical to any of these sequences.

9 An E2 polypeptide according to claim 8 selected from SEQ ID NO 2 or SEQ ID NO 4.

10 A method for producing an E2 polypeptide which method comprises culturing a transformed host cell comprising a polynucleotide as defined in claim 1 under conditions suitable for expression of the polypeptide.

11 An antibody specific for a polypeptide as defined in claim 9.

12 A method for identifying a chemical compound capable of modulating the activity of E2 which method comprises:
(i) contacting a chemical compound with an E2 polypeptide as defined in claim 1 or
(ii) a transgenic non-human mammal as defined in claim 6; and
(iii) measuring any effect of the chemical compound on the activity of the E2 polypeptide or the transgenic non-human mammal.

13 A method of making a pharmaceutical composition which comprises:
(i) the method for identifying a chemical compound according to claim 12;
(ii) mixing the compound thus identified with a pharmaceutically acceptable diluent or carrier.

14 A method according to claims 13 or 14 in which the chemical compound is for controlling insulin resistance syndrome and other related disorders such as non-insulin dependent diabetes mellitus (NIDDM), dyslipidemia, obesity and atherosclerosis.


SEQUENCE LISTING (21 sequences)

SEQ ID NO: 1 - DNA, 948 bp, Mus musculus
atggcttgcagtgagttttcctttcacatgccaagtctggaggagcttgctgaagttttgcagaaggggctaactgacaactttgctgatgtccaggtctcagtggttgactgcccagatttaacaaaggagccatttacctttcctgtaagaggcatctgtgggcaaactagaattgcagaagtaggaggtgtgccttacttattgcctcttgtaaacaaaaaaaaagtttatgacctaaatgaaattgcaaaagtaataaagctgcccggagcttttatccttggagcaggggcaggtccatttcagacgcttgggttcaattctgagttcatgccaattgttcaaacagcaagtgaacacaaccaacctgtgaatggaagttactttgcccataaaaaccctgcagatggagcgtgcctgctggagaaatacagccaaaaatatcatgattttggatgtgcactactggctaatctttttgccagtgagggccaacctggcaaggtcattgaagtgcaagccaagagaagaacaggagaacttaactttgtgagctgcatgagacagacactggaggagcactatggtgacaagcctgtggggatgggaggcaccttcattgtgcagaaggggaaagtgaaagcccacatcatgcctgcagagttttcttcctgcccactgaactcggacgaagctgtcaataaatggctacacttctatgagatgaaggctcccttggtttgtctaccagtttttgtttccaaagaccctggacttgatttgcgactggagcacacacatttcttcagtcatcatggagaaggtggacactaccactacgatacgaccccagacacagtggagtaccttggatacttctcacctgcacagttcctctatcgcattgatcagcccaaagagacccatgcctttgggagagattaa

SEQ ID NO: 2 - protein, 315 aa, Mus musculus
MACSEFSFHMPSLEELAEVLQKGLTDNFADVQVSVVDCPDLTKEPFTFPVRGICGQTRIAEVGGVPYLLPLVNKKKVYDLNEIAKVIKLPGAFILGAGAGPFQTLGFNSEFMPIVQTASEHNQPVNGSYFAHKNPADGACLLEKYSQKYHDFGCALLANLFASEGQPGKVIEVQAKRRTGELNFVSCMRQTLEEHYGDKPVGMGGTFIVQKGKVKAHIMPAEFSSCPLNSDEAVNKWLHFYEMKAPLVCLPVFVSKDPGLDLRLEHTHFFSHHGEGGHYHYDTTPDTVEYLGYFSPAQFLYRIDQPKETHAFGRD

SEQ ID NO: 3 - DNA, 948 bp, Homo sapiens
atggcttgtgctgagttttcttttcatgtaccaagtcttgaagagcttgctggagttatgcagaaggggttaaaagataactttgctgatgtccaggtctctgtagttgattgccctgatttgactaaggaaccctttacctttcctgtaaaaggcatctgtgggaaaactagaattgcagaagttggaggtgtgccttacttattgcctcttgtaaaccaaaaaaaagtttatgatctgaataaaattgcaaaagaaatcaagctgcctggagcctttattcttggagcaggagcaggtccatttcagactctcgggttcaattctgagtttatgccagttattcagacagaaagtgaacacaagcctcctgtaaatggaagttactttgcccatgtgaaccctgcagatggagggtgcctactggagaaatacagtgagaaatgtcatgattttcagtgtgcattactggctaatctttttgccagtgaaggccaacctggcaaggtaattgaggtgaaagccaaaagaagaactggaccacttaactttgtgacttgtatgagagagaccctggaaaaacattatggaaataagcctataggaatgggaggtactttcataattcagaagggaaaagtgaagtctcacattatgcctgcagaattttcttcctgccccttgaactctgatgaagaagtgaataaatggttgcatttttatgaaatgaaagctcctttggtttgtctaccagtttttgtctccagagacccagggtttgatttgcgactggagcacactcatttttttagtcgtcatggagaaggtggacactaccattatgacactactccagatatagtggaatatcttggatacttcttacctgcagagtttctctatcgcattgatcaaccaaaagagacgcattccattgggcgagattaa

SEQ ID NO: 4 - protein, 315 aa, Homo sapiens
MACAEFSFHVPSLEELAGVMQKGLKDNFADVQVSVVDCPDLTKEPFTFPVKGICGKTRIAEVGGVPYLLPLVNQKKVYDLNKIAKEIKLPGAFILGAGAGPFQTLGFNSEFMPVIQTESEHKPPVNGSYFAHVNPADGGCLLEKYSEKCHDFQCALLANLFASEGQPGKVIEVKAKRRTGPLNFVTCMRETLEKHYGNKPIGMGGTFIIQKGKVKSHIMPAEFSSCPLNSDEEVNKWLHFYEMKAPLVCLPVFVSRDPGFDLRLEHTHFFSRHGEGGHYHYDTTPDIVEYLGYFLPAEFLYRIDQPKETHSIGRD

SEQ ID NO: 5 - DNA, 1093 bp, Homo sapiens
cggactctgggtgttttgctaccgtgaccgtttagaaactgttcatacttggtgcgctgtggactcttgtgataattaaccaagagtagctctatttgtccaacctcacacctaaagaagaaagaaaatggcttgtgctgagttttcttttcatgtaccaagtcttgaagagcttgctggagttatgcagaaggggttataagataactttgctgatgtccaggtctctgtagttgattgccctgatttgactaagaaaccctttacctttcctgtaaaaagtttttttcttaggcatctgtgggaaagctagaattgcggaagttggaggtgtgccttacttattgcctcttgtaaaccaaaaaaaagtttatgatctgaataaaattgcaaaagaaatcaagctgcctggagcctttattcttggagcaggagcaggtccatttcagactctcgggttcaattctgagtttatgccagttattcagacagaaagtgaacacaagcctcctgtaaatggaagttactttgcccatgtgaaccctgcagatggagggtgcctactggagaaatacagtgagaaatgtcatgattttcagtgtgcattactggctaatctttttgccagtgaaggccaacctggcaaggtaattgaggtgaaagccaaaagaagaactggaccacttaactttgtgacttgtatgagagagaccctggaaaaacattatggaaataagcctataggaatgggaggtactttcataattcagaagggaaaagtgaagtctcacattatgcctgcagaattttcttcctgccccctgaactctgatgaggaagtgaataaatggttgcatttttatgaaatgaaagctcctttggtttgtctaccagtttttgtctccagagacccagggtttgatttgcgactggagcacactcatttttttagtcgtcatggagaaggtggacactaccattatgacactactccagatatagtggaatatcttggatacttcttacctgcagagtttctctatcgcattggtcaaccaaaagagacgcattccattgggcgagattaatcagc

SEQ ID NO: 6 - protein, 204 aa, Homo sapiens
MPVIQTESEHKPPVNGSYFAHVNPADGGCLLEKYSEKCHDFQCALLANLFASEGQPGKVIEVKAKRRTGPLNFVTCMRETLEKHYGNKPIGMGGTFIIQKGKVKSHIMPAEFSSCPLNSDEEVNKWLHFYEMKAPLVCLPVFVSRDPGFDLRLEHTHFFSRHGEGGHYHYDTTPDIVEYLGYFLPAEFLYRIGQPKETHSIGRD

SEQ ID NO: 7 - DNA, 16 bp, Artificial Sequence
  H-T11-A primer
aagcttttttttttta

SEQ ID NO: 8 - DNA, 16 bp, Artificial Sequence
  H-T11-C primer
aagctttttttttttc

SEQ ID NO: 9 - DNA, 16 bp, Artificial Sequence
  H-T11-G primer
aagctttttttttttg

SEQ ID NO: 10 - DNA, 13 bp, Artificial Sequence
  H-AP-1 primer
aagcttgattgcc

SEQ ID NO: 11 - DNA, 13 bp, Artificial Sequence
  H-AP-2 primer
aagcttcgactgt

SEQ ID NO: 12 - DNA, 13 bp, Artificial Sequence
  H-AP-3 primer
aagctttggtcag

SEQ ID NO: 13 - DNA, 13 bp, Artificial Sequence
  H-AP-4 primer
aagcttctcaacg

SEQ ID NO: 14 - DNA, 13 bp, Artificial Sequence
  H-AP-5 primer
aagcttagtaggc

SEQ ID NO: 15 - DNA, 13 bp, Artificial Sequence
  H-AP-6 primer
aagcttgcaccat

SEQ ID NO: 16 - DNA, 13 bp, Artificial Sequence
  H-AP-7 primer
aagcttaacgagg

SEQ ID NO: 17 - DNA, 13 bp, Artificial Sequence
  H-AP-8 primer
aagcttttaccgc

SEQ ID NO: 18 - DNA, 13 bp, Artificial Sequence
  H-AP-9 primer
aagcttcattccg

SEQ ID NO: 19 - DNA, 13 bp, Artificial Sequence
  H-AP-10 primer
aagcttccacgta

SEQ ID NO: 20 - DNA, 17 bp, Artificial Sequence
  Rgh primer
gacgcgaacgaagcaac

SEQ ID NO: 21 - DNA, 17 bp, Artificial Sequence
  Lgh primer
cgacaacaccgataatc